(19) United States
(12) Patent Application Publication
(10) Pub. No.: US 2005/0220754 A1
(43) Pub. Date: Oct. 6, 2005

(54) Vitamin directed targeting therapy

(75) Inventors: Gregory Russell-Jones, Middle Cove (AU); John Fergus McEwan, Blakehurst (AU)

Correspondence Address: FOLEY AND LARDNER;SUITE 500, 3000 K STREET NW, WASHINGTON, DC, 20007, US

(73) Assignee: BIOTECH AUSTRALIA PTY. LTD.

(21) Appl. No.: 11/127,285

(22) Filed: May 12, 2005

Related U.S. Application Data
(63) Continuation of application No. 09/805,506, filed on Mar. 14, 2001.
Application No. 09/805,506 is a division of application No. 09/585,282, filed on Jun. 2, 2000, now abandoned.
(60) Provisional application No. 60/137,449, filed on Jun. 2, 1999.

(51) Int. Cl. A61K 38/17; A61K 38/16

(52) U.S. Cl. 424/78.3; 514/12; 514/17; 514/18

(57) ABSTRACT

The invention relates to vitamin-mediated targeting for the delivery of agents and active substances in the therapy of disease. Targeting using vitamins essential for cancer growth are used in complexes for amplified delivery of cytotoxic drugs to tumors and cancer cells, with a concomitant reduction in toxicity to the subject being treated.

This application is a continuation of U.S. patent application Ser. No. 09/805,506, filed Mar. 14, 2001, which is a divisional of U.S. patent application Ser. No. 09/585,282, filed Jun. 2, 2000, which claims priority to U.S. Provisional Patent Application No. 60/137,449, filed Jun. 2, 1999, which are all incorporated by reference in their entirety.

### TECHNICAL FIELD

The invention relates to vitamin-mediated targeting for the delivery of agents and active substances in the therapy of disease. More particularly the invention relates to combined targeting using vitamins essential for cancer growth in vitamin complexes used to deliver cytotoxic drugs to tumors and cancer cells. The invention also relates to processes for preparing the complexes, pharmaceutical compositions containing same, methods of treatment involving the complexes and uses of the complexes in the manufacture of medicaments.

### BACKGROUND OF THE INVENTION

Chemotherapy has been, for many decades, one of the major approaches to the control and cure of malignant neoplasms (cancers). In conventional cancer chemotherapy it is often necessary to increase the quantity of cytotoxic drugs administered in an exponential fashion in order to obtain a linear increase in the death of cancer cells. This in turn leads to an undesirable increase in non-specific cytotoxicity of bystander, healthy cells. Hence it is often necessary to repeatedly deliver smaller doses of cytotoxin, which inevitably leads to the survival of a small fraction of drug-resistant cells. This in turn has necessitated the development of more specific and stronger cytotoxic drugs, however their nonselective action on cells other than cancerous cells still remains a major problem.

In an attempt to increase the dose of cytotoxic agent delivered to the tumor cell, specific targeting agents such as monoclonal antibodies to “tumor-specific antigens” have been employed. While this therapy has proven to produce more specific targeting, in many instances it has not been possible to deliver enough cytotoxic agent coupled to the antibody for effective tumoricidal activity.

Recently focus has switched to the use of molecules essential for growth to be used as targeting agents. In particular, research has concentrated on the use of vitamins and in particular folic acid and vitamin B₁₂ (cobalamin, Cbl, VB₁₂) for tumor specific targeting.

Folic acid enters cells either through a carrier protein, termed the reduced folate carrier, or via receptor-mediated endocytosis facilitated by folate receptors (FR). Folate receptors are significantly over-expressed on a large proportion of human cancer cells including ovarian, breast, lung, endometrial, renal, colon, and cancers of myeloid hematopoietic cells. There are two folate receptors, FR-α, and FR-β. In general FR-α is upregulated in malignant tissues of epithelial origin such as ovarian carcinoma, while FR-β is overexpressed in malignant tissues of non-epithelial origin. While the FR have been detected in normal tissues involved in the retention and uptake of the vitamin, these tissues are in protected sites and generally not accessible following blood-borne delivery of folate conjugates. Thus there is expression in the choroid plexus, the intestinal brush border apical membrane surface and the proximal tubules of the kidney. In the latter case the receptor probably functions to scavenge excreted folate, and as such would not be accessible to large molecular weight folate complexes. Folate-mediated tumor targeting has been exploited to date for delivery of the following molecules and molecular complexes (i) protein toxins, (ii) low-molecular-weight chemotherapeutic agents, (iii) radio-imaging agents (iv) MRI contrast agents, (v) radiotherapeutic agents, (vi) liposomes with entrapped drugs, (vii) genes, (viii) antisense oligonucleotides, (ix) ribozymes, and (x) immunotherapeutic agents.

Cellular uptake of vitamin B₁₂ (cobalamin, Cbl, VB₁₂) is mediated primarily by the plasma protein transcolbamin II (TCII). Following binding of Cbl to TCII the resultant TCII-Cbl complex binds with high affinity to receptors on the surface of cells and is internalized by the cell via a process called receptor-mediated endocytosis (RME). Once inside the cell the Cbl is enzymatically modified to form two coenzymes, which are in turn for two essential metabolic pathways. One involves the methylation of homocysteine in the de novo synthesis of methionine, and is catalyzed by methionine synthetase. The other pathway involves the rearrangement of methyl malonyl CoA to succinyl CoA, and is catalyzed by methyl malonyl CoA mutase. It has recently been shown that the in vitro proliferation of human and murine leukemia cells is dependent upon both TCII and Cbl (see McLean, G. R., Quadros, E. B., Rothenberg, S. P., Morgan, A. C., Schrader, J. W., and Ziltener, H. J., Antibodies to transcobalamin II block in vitro proliferation of leukemic cells, Blood, 1997, 89, 235-242). Several workers have now concentrated on utilizing Cbl conjugates for both radio-imaging and for targeted cancer chemotherapy (see Smeltzer, C. C., Pinson, P. R., Munger, J. M., West, F. G., and Grissom, C. B., Cytotoxicities of two new cobalamin bioconjugates, Proceedings Ninth International Symposium on Recent Advances in Drug Delivery Systems, 1999, pp 232-3; Canon, M. J., Munger, J. M., West, F. G., and Grissom, C. B., Synthesis and uptake of radiolabeled cobalamin bioconjugate, Proceedings Ninth International Symposium on Recent Advances in Drug Delivery Systems, 1999, pp 230-1; and Pinson, P. R., Munger, J. M., West, F. G., and Grissom, C. B., Synthesis of two doxorubicin-cobalamin bioconjugates, Proceedings Ninth International Symposium on Recent Advances in Drug Delivery Systems, 1999, pp 228-9).

However, there is one major limitation to the use of vitamins as molecules to target to tumor cells, and that is the targeting of each molecule is not entirely specific for tumor cells. For example the majority of folate-drug complexes are very small and as such are excreted in the kidneys and re-absorbed in the proximal tubules, thus leading to undesirable accumulation of folate-drug complexes in the kidney. Similarly for VB₁₂-drug complexes there is undesirable accumulation in the liver.

Limitations of the vitamin complexes are partly addressed by the production of large molecular weight polymer complexes between the vitamin and the agent or active substance to be delivered. Thus, amplification of drug delivery can occur by linkage of a drug/pharmaceutical to a bio-compatible polymer backbone to which a number of vitamin molecules are linked, either subsequently, previously or concurrently. Preferably the linkage to the polymer, or the polymer to which the pharmaceutical is linked, should be biodegradable. This strategy is the subject of a folate-polymer complex patent application (PCT/AU00/00406, filed 4 May 2000) and a VB₁₂-polymer complex patent (Russell-Jones et al., 1995 U.S. Pat. No. 5,449,720) both specifications of which are incorporated herein in their entirety by reference. Alternatively the drug could be included within a vitamin-coated nanoparticle, which strategy is the subject of a folate-nanoparticle patent application (PCT/AU00/00405, filed 4 May 2000) and a VB₁₂-nanoparticle patent (Russell-Jones et al., 1997 EP 0,531,497 B1) both specifications of which are incorporated herein in their entirety by reference. Nonetheless there is still a need for better and more effective drug targeting to sites such as tumor tissues and cancer cells.

Thus, it is an object of the present invention to overcome, or at least alleviate one or more of the above mentioned disadvantages of the prior art.

### SUMMARY OF THE INVENTION

Accordingly, the present inventors have found that amplification of agent or active substance delivery can be enhanced by the use vitamin complexes. By administering vitamin-complexes it is possible to target and deliver greater amounts of agents and active substances such as drugs and pharmaceuticals to tumor tissues and cancer cells, whilst at the same time minimizing the unwanted accumulation of these agents at other sites or in organs, such as the kidneys and liver. This greatly reduces the toxicity of the drug or pharmaceutical being delivered and amplifies drug/pharmaceutical delivery to the tissues and cells.

Thus, according to one embodiment there is provided a complex having the general formula
(TA-Q)ₙ-P-(Q′-A)ₘ
wherein, TA is selected from the group of biotin, a biotin derivative, and a biotin analog, n is the molar substitution ratio of TA in the complex, and is a number from 1.0 to 50, P is a pharmaceutically acceptable polymer, A is a pharmaceutically active substance, m is a molar substitution ratio of A in the complex, and is a number in the range from 1.0 to about 1000, and Q and Q′ are independently a covalent bond, or a spacer compound linking TA, P and A by a linkage, wherein the linkage is not a thiol-cleavable linkage.

Another embodiment provides a method for making a complex having the general formula
(TA-Q)ₙ-P-(Q′-A)ₘ
wherein, TA is selected from the group of biotin, biotin derivative, and biotin analog, n is the molar substitution ratio of TA in the complex, and is a number from 1.0 to 50, P is a pharmaceutically acceptable polymer, A is a pharmaceutically active substance, m is a molar substitution ratio of A in the complex, and is a number in the range from 1.0 to about 1,000, Q and Q′ are independently a covalent bond, or a spacer compound linking TA, P and A by covalent bonds, and wherein TA, P, and A are joined by a linkage, wherein the linkage is not a thiol-cleavable linkage comprising one or more of the following steps:
(a) reacting A with P to form an intermediate A-P complex;
(b) chemically modifying the A to provide at least one functional group capable of forming a chemical linkage, and reacting A and P to form the intermediate A-P complex;
(c) chemically modifying TA to provide at least one functional group capable of forming a chemical linkage and reacting TA and P to form an intermediate TA-P complex;
(d) chemically modifying A and P to provide functional groups capable of forming a chemical linkage, and reacting A and P to form an intermediate A-P complex;
(e) reacting A with at least one cross-linking agent and reacting A with P to form a intermediate A-P complex;
(f) reacting TA with at least one cross-linking agent and reacting P and a carrier to form an intermediate TA-P complex;
(g) reacting A and P with at least one cross-linking agent and reacting the A and P to form an intermediate A-P complex;
(h) reacting the A directly with a polymeric support to form an intermediate containing one or more molecules of the A linked to P to form a support-bound A-P intermediate complex, and subsequently coupling the support-bound A-P intermediate complex to one or more TA; and
(i) coupling one or more TA to a polymeric support and subsequently reacting support-bound TA intermediate with one or more molecules of the A to yield a complex containing one or more molecules of A.

Yet another embodiment provides a method of making the pharmaceutical composition, comprising mixing the complex together with a pharmaceutically acceptable carrier, excipient, diluent or adjuvant or a combination thereof.

Another embodiment provides a method for the treatment, prophylaxis or amelioration of a disease, comprising administering to a subject in need thereof a therapeutically effective amount of a complex having the general formula
(TA-Q)ₙ-P-(Q′-A)ₘ
wherein, TA is selected from the group of biotin, biotin derivative, and biotin analog; n is the molar substitution ratio of TA in the complex, and is a number from 1.0 to 50, P is a pharmaceutically acceptable polymer, A is a pharmaceutically active substance, m is a molar substitution ratio of A in the complex, and is a number in the range from 1.0 to about 1000, Q and Q′ are independently a covalent bond, or a spacer compound linking TA, P and A by covalent bonds, and wherein TA, P, and A are joined by a linkage, wherein the linkage is not a thiol-cleavable linkage.

According a further embodiment, there is provided a kit for the delivery of agents or active substances to a subject, A kit for the delivery of one or more agents to a subject comprising a complex of the invention.

It is to be understood that both the foregoing general description and the following detailed description are exemplary and explanatory only, and are not restrictive of the invention as claimed.

### BRIEF DESCRIPTION OF THE FIGURE

FIG. 1 represents the targeting of a tumor with a pharmaceutical composition of the present invention comprising a Cbl-targeting complex and a folate-targeting complex. In FIG. 1, a subject is depicted in which the star (1) represents a tumor, the crescent (2) represents the liver and the ovals (3) represent the kidneys.

FIG. 1A shows the targeting of a tumor with a Cbl complex and the unwanted accumulation of the Cbl complex in the liver.

FIG. 1B shows the targeting of a tumor with a folate complex and the unwanted accumulation of the folate complex in the kidneys.

FIG. 1C shows the dual targeting of a tumor with a folate complex/Cbl complex composition of the present invention. It can be seen in FIG. 1C that there is an amplification of agent or drug/pharmaceutical delivery to the tumor with respect to the delivery to either the kidneys or liver, which in turn leads to a relative reduction in the toxicity of the cytotoxic complexes to the subject being treated.

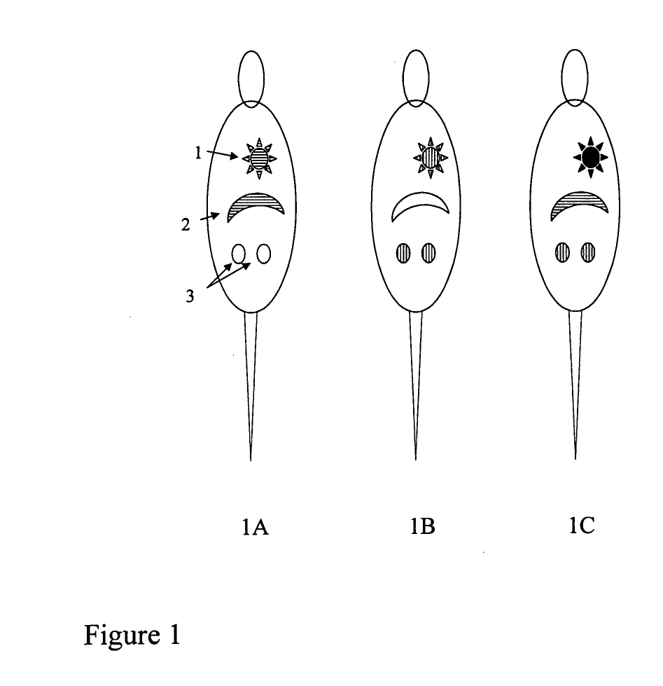

### DETAILED DESCRIPTION OF THE PREFERRED EMBODIMENTS

An advantage provided by the present invention is the ability to amplify agent or active substance delivery to a subject by the use of vitamin complexes. The vitamin complexes preferentially target tumor tissues and cancer cells for it is in these tissues and cells that there often is an upregulation of vitamin receptors. Vitamins suitable for making vitamin complexes include, amongst others, folic acid, VB₁₂, riboflavin and biotin, more preferably folic acid and VB₁₂. It will be understood that derivatives and analogues of vitamins are also within the scope of the present invention. Analogues contemplated herein include, but are not limited to, modification to the ring structure, functional groups or side chains of the vitamin molecule including the additional removal of protecting groups and salts and complexes thereof derived from any source such as being chemically synthesized or identified by screening process such as natural product screening provided that the analogue possesses some binding activity for the vitamin receptor. It will be understood by those skilled in the art that upregulated receptors other than just the vitamin receptors on tumor or cancer cells can be targeted by the complexes of the present invention.

Administration of the pharmaceutical compositions of the present invention is either by the simultaneous or sequential administration of vitamin complexes, preferably a folate complex and a VB₁₂ complex. The vitamin complexes deliver agents or active substances, in particular hormones, drugs, prodrugs, enzymes, proteins, peptides, toxins, immunogens or DNA or RNA analogues to subjects.

Suitable toxins for use in the invention include, but are not limited to, ricin, abrin, diphtheria toxin, modecin, tetanus toxin, mycotoxins, mellitin, α-amanitin, pokeweed antiviral protein, ribosome inhibiting proteins, especially those of wheat, barley, corn, rye, gelonin and maytansinoid.

Suitable cytotoxic agents for use in the invention include, but are not limited to, alkylating agents such as chlorambucil, cyclophosphamide, melphalan, cyclopropane; anthracycline antitumor antibiotics such as doxorubicin, daunomycin, adriamycin, mitomycin C, 2-(hydroxymethyl)anthraquinone; antimetabolites such as methotrexate, dichloromethatrexate: cisplatin, carboplatin, and metallopeptides containing a metal selected from the group consisting of platinum, copper, vanadium, iron, cobalt, gold, cadmium, zinc and nickel. Other agents include DON, thymidine, pentamethylmelamin, dianhydrogalactitol, 5-Methyl-THF, anguidine, maytansine, neocarzinostatin, chlorozotocin, AZQ, 2′deoxycoformycin, PALA, AD-32, m-AMSA and misonidazole.

In one embodiment of the invention different vitamins are conjugated to the same type of active substance. Administration of the vitamin complexes so produced results in the complexes targeting cancer cells which are upregulated for those vitamins whilst at the same time unwanted accumulation of the vitamin complexes is spread over different organs and tissues which also happen to be targeted by the particular vitamins. This results in the amplification of delivery of active substances, such as cytotoxins, to the target cells thereby greatly reducing the toxicity of the cytotoxic conjugates to other parts of the body.

In another embodiment different vitamins are conjugated to different active substances. This also results in the amplification of, for example, cytotoxic agents to target cells in combination drug therapy.

In an important embodiment of the present invention it is possible to administer a first vitamin conjugate with a relatively non-toxic prodrug of a cytotoxic agent together with or sequentially to a second vitamin conjugate with a relatively non-toxic enzyme. The first vitamin conjugate may for example use folic acid as the targeting vitamin. The folate moiety targets the tumor tissue to be treated and also happens to target the kidneys, delivering the prodrug rather than the cytotoxic agent itself. Sequential or simultaneous administration of the second vitamin conjugate using for example a VB₁₂-enzyme complex will target the tumor and the liver, as a result of the VB₁₂ targeting. The enzyme is adapted to transform the prodrug into a cytotoxic agent which directly acts on the targeted tumor tissue. The toxicities of the administered vitamin complexes are greatly reduced because substantially less cytotoxic drug will be synthesized in other tissues and organs in the treated subject. This is a result of some accumulation in the kidneys of relatively non-toxic folate-prodrug complex but little or no delivery of the VB₁₂-enzyme complex, whilst there is some accumulation of the relatively non-toxic VB₁₂-enzyme in the liver but little or no delivery of the folate-prodrug complex.

Most preferably the subject firstly receives a dose of an antibody-enzyme conjugate, after which the patient then receives a dose of a prodrug conjugate. A cytotoxic agent is then synthesised or released from the prodrug conjugate at the targeted tumor site by the action of the enzyme.

In a further embodiment of the present invention, the agents or active substances may be delivered by existing encapsulation methods or polymer-drug complexes. Suitable encapsulation methods utilising polymers in the preparation of biodegradable nanoparticles and microspheres, which has the targeting molecule attached to it, are described in the above mentioned folate-nanoparticle patent application (PCT/AU00/00405, filed 4 May 2000) and the VB₁₂-nanoparticle patent (Russell-Jones et al., 1997 EP 0,531,497 B1) and are described below as follows:—

Polymers suitable for the formation of nanoparticles by solvent evaporation (in liquid drying) include, amongst others, poly-lactic acid, poly-(Lactide/co-glycolide), poly-hydroxybutyrate, poly-hydroxyvalerate, poly-(hydroxybutyrate/valerate), ethyl cellulose, dextran, polysaccharides, polyalkylcyanoacrylate, poly-methyl-methacrylate, poly(e-caprolactone) and various combinations and co-polymers of the above.

Polymers suitable for the formation of microspheres by interfacial precipitation/polymerization include, amongst others, EUDRAGIT™; Poly(N^{a},N^{e}-L-lysinediylterephthaloyl); polymers formed by the reaction of Lysine hydrochloride and p-phthaloyl dichloride; by the reaction of acryloylated maltodextrin or acryloylated hydroxyethyl starch with ammonium peroxodisulfate and N,N,N′,N′-tetramethylethylenediamine. Microspheres can also be formed by the polymerization of various diamines such as ethylene diamine, phenylenediamine, toluene diamine, hexamethylene diamine, or diols such as ethylene diol, bisphenol, resorcinol, catechol, pentanediol, hexanediol, dodecanediol, 1,4-butanediol, with diacid chlorides such as sebacoylchloride and adipoyl chloride, or diisocynates such as hexamethylene diisocyanate using the methods fully described in EP 85870002.4.

Polymers suitable for the formation of microspheres by polymer phase separation include co-poly(vinyl chloride:vinyl alcohol:vinyl acetate), cellulosic polymers, polyvinyl acetate, polyvinyl alcohol, polyvinylchloride, natural and synthetic rubbers, polyacrylates, polystyrene and the like. Methods to synthesize such microspheres are fully described in U.S. Pat. No. 4,166,800.

Polymers suitable for the formation of microspheres by complex coacervation include, amongst others, mixtures of polyanions, such as gum arabic, alginate, carboxymethyl cellulose, carboxymethyl starch, polystyrene sulfonic acid, polyvinyl sulfonic acid, poly-d-glucuronic acid, poly-pyruvic acid, carrageenan, heparin sulfate, polyphosphate with polycations, such as polylysine, gelatin.

Polymers suitable for the formation of microspheres by polymer/polymer incompatibility include, amongst others, ethyl cellulose, ethylene vinyl acetate polymer, poly(lactide), or poly(vinylidene chloride) mixed with polymers such as polyethylene, silicone, polyisobutylene or polybutadiene.

Other materials suitable for formation of microspheres include, starch, cross-linked albumen, polyacrylamide, cross-linked gelatin and others known to those skilled in the art of microsphere preparation.

Suitable polymer-drug complexes utilize targeting molecules to deliver agents or active substances as described in the above mentioned folate-polymer complex patent application (PCT/AU00/00406, filed 4 May 2000) and the VB₁₂-polymer complex patent (Russell-Jones et al., 1995 U.S. Pat. No. 5,449,720) and are described below as follows:—

Polymers useful according to the invention include potentially biodegradable polymers such as dextran and its derivatives, amino acid polymers such poly-lysine, poly-glutamic acid. Non-biodegradable polymers include poly[N-(2-hydroxypropyl)-methacrylamide], to which is attached biodegradable side chains such as those containing ester linkages, or amino acid sequences cleavable within lysosomal vacuoles, i.e. Gly-Phe-Leu-Gly (see Rihova, B. and Kopecek J., Biological properties of targetable poly[N-(2-hydroxypropyl)-methacrylamide]-antibody complexes, J. Control Rel., 1985, 2:289-310). Other amino acid spacers cleavable by intracellular proteases include Gly-Phe-Ala; Gly-Phe-Ala-Gly; Gly-Phe-Tyr-Ala; and Gly-Phe-Tyr-Ala-Ala (see Rejmanova, P., Obereigner, B., and Kopecek, J., Makromol. Chem., 1981, 182:1899-1915).

The vitamin targeting agents can also be linked to polymers to which are linked various enzymes, drugs, and cytotoxic agents for the control of tumor growth. These targeting agent-polymer-drug conjugates are suitable for parenteral delivery to tumors as they can utilize the aforementioned targeting agent (TA) receptor system for uptake binding and uptake, and have the added advantage of increasing the amount of pharmaceutical agent which can be delivered via the TA uptake mechanism. A further advantage of using polymers in combination with folic acid, or an analogue thereof, as a TA, is their ability to avoid or at least reduce targeting to the kidneys by virtue of their size.

In one embodiment of the invention the linkage joining the pharmaceutical, or the TA to the polymer is a disulfide bond. In a further embodiment of the invention the linkage joining the pharmaceutical, or the TA to the polymer is an ester linkage. In yet another embodiment of the invention the linkage joining the pharmaceutical or the TA to the polymer is a γ-glutamyl-ε-lysine bond. In yet another embodiment of the invention, the linkage joining the pharmaceutical or the TA to the polymer is a diazo-linkage.

In a preferred embodiment vitamin polymer complexes have the general formula:
(TA-Q)ₙ-P-(Q′-A)ₘ
wherein, TA, is either vitamin B₁₂ or an analogue thereof which will bind via TCII to surface receptors on tumor cells, or where TA is folate or an analogue thereof which will bind via folate receptors to surface receptors on tumor cells, and where
n, the molar substitution ration of TA in the complex, is in the range from 1.0 to 50.0, and
P is a pharmaceutically acceptable polymer, and
A is a pharmaceutically active substance, and
m, the molar substitution ratio of A in the complex, is in the range from 1.0 to 1000, and
Q and Q′ are independently a covalent bond, or a spacer compound linking folate, P and A by covalent bonds.

The polymer, P, of the present invention can be any pharmaceutically acceptable polymer. The polymer is able to attach to at least one carrier molecule and to at least one, but preferably a multiplicity of active substance molecules.

Suitable polymers for substitution with folate and modification according to the invention, include poly[N-(2-hydroxypropyl)-methacrylamide], dextran, chondroitan sulfate, water soluble polyurethanes formed by covalent linkage of PEG with lysine, poly(glutamic acid), poly(hydroxypropyl glutamine) and branched chain polypeptides formed by the dual modification of the α- and ε-amino groups of lysine during the peptide synthesis. Such polymers may have multiple amino-termini, to which can be conjugated a plurality of the pharmaceutical or drug to be delivered. The polymers can also be formed with multiple cystines, to provide free thiols, or multiple glutamates or aspartates, to provide free carboxyls for conjugation using suitable carbodiimides. Similarly the polymer can contain multiple histidines or tyrosines for conjugation.

Folate is most easily covalently attached to a ligand, or the polymer, via either its α or γ carboxylic acid moiety. It has been shown, however, that the α-carboxyl derivatives have low avidity for the folate receptor, whereas the γ-carboxy derivatives have similar affinity to native folate.

The spacer compounds Q and Q′ are optional. When they are absent the carrier (folate or a suitable derivative of vitamin B₁₂) and/or the active substance A are linked to polymer P by a direct covalent bond. They are introduced either to improve the receptor affinity of the carrier complex or to overcome problems in the coupling of the carrier, and/or the active substance A arising from unfavourable steric interactions between the carrier and A with the polymer P, or to increase the bioactivity of A in the complex. The spacer compounds may also act as linking agents, being bi-functional compounds with selected functional groups on each end to react with suitable functional groups located on the polymer, and also on the vitamin carrier molecule and/or on the pharmaceutically active substances.

Polymers to which are conjugated various cytotoxic drugs have been previously described. These polymers have been targeted to cancer cells using for example specific antibodies or sugar moieties. Once the drug-polymer has reached its target tissue the complex is endocytosed by the target cell and the pendant drug is released by the action of lysosomal enzymes, or by cleavage of a disulfide-linked drug by intracellular glutathione.

It will be evident to a person skilled in the art that different variations of vitamin conjugate pairs can be used provided that different vitamins target and deliver, either directly or indirectly, agents or active substances to the same site to be treated. Preferred vitamin complex pairs include, amongst others, the following:
a) Cbl-enzyme+Folate-substrate-cytotoxin;
b) Folate-enzyme+Cbl-substrate-cytotoxin;
c) Cbl-enzyme+Folate-Polymer-substrate-cytotoxin;
d) Folate-enzyme+Cbl-Polymer-substrate-cytotoxin;
e) Cbl-enzyme+Folate-NP-substrate-cytotoxin;
f) Folate-enzyme+Cbl-NP-substrate-cytotoxin; and
g) Cbl-NP-Enzyme+Folate-NP-substrate-cytotoxin.

Particularly preferred enzyme prodrug combinations suitable for the invention include the following peptidase substrate pairs:
Carboxypeptidase G2 in combination with prodrugs of nitrogen mustard alkylating agents (Bagshawe, K. C. 1987 Br J Cancer, 56, 531; Bagshawe, K. C., Springer, C. M., Searle, F., Antoniw, P., Sharma, S. K., Melton, R. G., Sherwood, R. F. 1988 Br. J. cancer, 58, 700; Searle, F., Bier, C., Buckley, R. G., Newman, S., Pedley, R. B., Bagshawe, K. D., Melton, R. G., Alwan, S. M., Sherwood, R. F. 1986, Br. J. Cancer, 53, 377; Springer, C. J., Antoniw, P., Bagshawe, K. C., Searle, F., Bisset, G. M. F., Jarman, M. 1990 J. Med. Chem., 33, 677).
Carboxypeptidase A or B and mono- or dipeptide prodrugs of methotrexate, such as alanyl-MTX.
Penicillin V/G amidase in combination with p-hydroxyphenoxy-derivatives of doxorubicin and melphalan.
Penicillin G amidase in combination with phenylacetamides of doxorubicin, melphalan and palytoxin.
β-Lactamases and derivatives of cephalosporins and methotrexate, or cephalosporins and 5-fluorouracil, or alternatively cephalosporins and analogues of vinblastine. Others include prodrugs formed with cephalosporins and doxorubicin, carboplatin analogues and taxol.
Alkaline phosphatase and phosphate prodrugs of etoposide (etoposide phosphate), mitomycin C (mitomycin phosphate), phenol mustard, nitrogen mustard, and doxorubicin (doxorubicin phosphate) (Senter, P. D. 1990 Activation of prodrugs by antibody-enzyme conjugates: a new approach to cancer therapy. FASEB J., 4, 188-193).

Further preferred enzyme prodrug combinations suitable for the invention include the following:
β-Glucuronidase and glucuronic acid derivatives of nitrogen mustard analogues, phenol mustard analogues.
α-Galactosidase and glycosidic derivatives of daunorubicin.
Cytosine deaminase in combination with 5-fluorocytosine.
Nitroreductase and 5-(aziridin-1-yl)-2,4-dinitrobenzamide, actinomycin, or mitomycin.
Arylsulfatase and sulfate derivatives of etoposide or phenol mustard.
β-Galactosidase and glycosidic derivatives of daunorubicin.

The vitamin complexes used in the present invention may also be formed from more than one active substance linked to a polymer, which is linked to at least one targeting agent (vitamin). The ability of the targeting agent to undergo the binding reactions necessary for uptake and transport of the active substance in a vertebrate host and the activity of the active substance are substantially maintained, following conjugation or following biological release of the active substance from the polymer. Suitable methods for the manufacture of these vitamin complexes comprises one or more of the following steps:
a) reacting the active substance with the polymer to form said complex;
b) chemically modifying the active substance to provide at least one functional group capable of forming a chemical linkage, and reacting the active substance and polymer to form said complex;
c) chemically modifying the targeting agent to provide at least one functional group capable of forming a chemical linkage and reacting the targeting agent and polymer to form said complex;
d) chemically modifying the active substance and the polymer to provide functional groups capable of forming a chemical linkage, and reacting the active substance and polymer to form said complex;
e) reacting the active substance with at least one cross-linking agent and reacting the active substance of polymer to form said complex;
f) reacting the targeting agent with at least one cross-linking agent and reacting the polymer and carrier to form said complex; g) reacting the active substance and polymer with at least one cross-linking agent and reacting the active substance and polymer to form said complex;
h) reacting the active substance directly with a polymeric support to form an intermediate containing one or more molecules of the active substance linked to the polymer, and subsequently coupling the polymer-active substance intermediate to one or more targeting agents; and
i) coupling one or more targeting agents to a polymeric support and subsequently reacting the targeting agent-polymer intermediate with one or more molecules of the active substance to give a final complex containing one or more molecules of the active substance.

The above method may also include modification of the polymeric support to introduce functional groups capable of reacting either directly with the active substance or with a chemically-modified form of the active substance. The resulting polymer-active substance intermediate contains one or more molecules of the active substance, said intermediate being suitable for coupling to the targeting agent to give a complex capable of amplified delivery of the active substances.

Suitable extended spacers for the conjugation of the pharmaceutical, vitamin B₁₂ or folate to the polymer matrix include: disuccinimidyl suberate (DSS), bis(sulfosuccinimidyl) suberate (BSS), ethylene glycolbis(succinimidylsuccinate) (EGS), ethylene glycolbis(sulfosuccinimidylsuccinate) (Sulfo-EGS), p-amino-phenylacetic acid, dithiobis(succinimidylpropionate) (DSP), 3,3′-dithiobis(sulfosuccinimidylpropionate) (DTSSP), disuccinimidyl tartarate (DST), disulfosuccinimidyl tartarate (Sulfo-DST), bis[2-(succinimidyloxycarbonyloxy)-ethylene]sulfone (BSOCOES), bis[2-(sulfosuccinimidooxycarbonyloxy)-ethylene]sulfone (Sulfo-BSOCOES), dimethyl adipimidate.2 HCl (DMA), dimethyl pimelimidate.2 HCl (DMP), dimethyl suberimidate.2 HCl (DMS), N-succinimidyl(4-iodoacetyl)aminobenzoate (SIAB), and succinimidyl 4-(p-maleimidophyl)butyrate (SMPB).

Suitable cross-linking agents for use in the preparation of thiol-cleavable biodegradable linkers include: N-succinimidyl 3-(2-pyridyldithio)propionate (SPDP), iminothiolane, sulfosuccinimidyl 6-[3-(2-pyridyldithio)propionamido]hexanoate (Sulfo-LC-SPDP), succinimidyl 6-[3-(2-pyridyldithio)propionamido]hexanoate (LC-SPDP), sulfosuccinimidyl 6-[α-methyl-α-(2-pyridyldithio)toluamido]hexanoate (Sulfo-LC-SMPT), 1,4-di[3′-(2′-pyridyldithio)propionamido]butane (DPDPB), 4-succinimidyloxycarbonyl-α-methyl-α-(2-pyridyldithio)-toluene (SMPT), and dimethyl 3,3′dithiobispropionimidate.2 HCl (DTBP).

Suitable analogues of VB₁₂ for derivatization prior to conjugation to the polymer include any variant or derivative of VB₁₂ (cyanocobalamin) which possesses binding activity to intrinsic factor. Preferred analogues of VB₁₂ also include aquocobalamin, adenosylcobalamin, methylcobalamin, hydroxycobalamin, cyanocobalamin, carbanalide, and 5-methoxybenzylcyanocobalamin [(5-MeO)CN-Cbl] as well as the desdimethyl, monoethylamide and the methylamide analogues of all of the above. Other analogues include all alkyl cobalamins in which the alkyl chain is linked to the corrin nucleus by a direct CoC covalent bond. Other analogues include chlorocobalamin, sulfitocobalamin, nitrocobalamin, thiocyanatocobalamin, benzimidazolecyanocobalamin derivatives such as 5,6-dichlorobenzimidazole, 5-hydroxybenzimidazole, trimethylbenzimidazole, as well as adenosylcyanocobalamin [(Ade)CN-Cbl], cobalamin lactone, cobalamin lactam and the anilide, ethylamide, monocarboxylic and dicarboxylic acid derivatives of VB₁₂ or its analogues.

Preferred derivatives of VB₁₂ also include the mono-, di- and tricarboxylic acid derivatives or the propionamide derivatives of VB₁₂. Targeting molecules may also include analogues of VB₁₂ in which the cobalt is replaced by zinc or nickel. The corrin ring of VB₁₂ or its analogues may also be substituted with any substituent which does not effect its binding to IF, and such derivatives of VB₁₂ or its analogues are part of this invention. Other derivatives of VB₁₂ or its analogues which have a functional group which is able to react with the spacer compound are also part of the invention. Other derivatives and analogues of VB₁₂ are discussed in Schneider, Z. and Stroinski, A., Comprehensive VB₁₂; (Walter De Gruyter; Berlin, NY; 1987), the disclosure of which is incorporated herein by reference.

Still other derivatives of VB₁₂ include those in which the 5′-hydroxyl group of the ribose moiety of the nucleotide ligand is modified. These derivatives include, but are not limited to derivatives formed by reaction with succinic anhydride, glutaric anhydride, p-maleimidophenyl isocyanate, oxirane, benzoquinone or cyanuric chloride. Alternatively derivatives can be formed by activation with 1,1′-carbonyldiimidazole and subsequent reaction with diamino-spacers, amino-acid-spacers, or alternatively with amino-alkyl chains to form hydrophobic derivatives (see Vitamin B₁₂ derivatives and methods for their production, PCT/AU99/00462, filed 11 Jun. 1999, Russell-Jones, G. J., and McEwen, J. F.).

Reference to the term “folate” as used herein is to be considered in its broadest context and refers to the carboxylic acid anion of folic acid and, where not stated, the counter cation may be any suitable cation including pharmaceutically acceptable cations and may also include a proton, i.e. folic acid. The term “folate” may be taken to include reference to analogues of the folate molecule, such as methotrexate, and preferably where the analogue possesses some binding activity for the folic acid receptor.

Reference herein to “treatment” and “prophylaxis” is to be considered in its broadest context. The term “treatment” does not necessarily imply that a host is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of onset of a particular condition. “Treatment” may also reduce the severity of an existing condition.

The subject of the treatment or prophylaxis is a vertebrate host, preferably a veterinary, domestic or agricultural animal or human, or more preferably a mammal, such as, but not limited to, human, primate, livestock animal (e.g. sheep, cow, horse, donkey, pig), companion animal (e.g. dog, cat), laboratory test animal (e.g. mouse, rabbit, rat, guinea pig, hamster), or a captive wild animal (e.g. fox, deer). Preferably the mammal is a human or primate. Most preferably the mammal is a human.

It will be understood that those skilled in the art will be able to employ method commonly known in the art for preparing suitable medicaments in concentrations and presented in forms appropriate to the administration of the folate complexes of the invention, optionally with other active agents as required, in suitable treatment regimes to achieve the desired physiological effect on the vertebrate host to be treated.

In accordance with these methods, the agents herein defined may be co-administered with one or more other compounds or molecules. For example, the pharmaceutical compositions of the invention may be administered in combination with other chemotherapeutic agents or other ameliorative active substances. “Administered in combination” means simultaneous administration in the same formulation or in two different formulations via the same or different routs or sequential administration by the same or different routes. “Sequential administration” means a time difference of from seconds, minutes, hours or days between the administration of the formulations. These agents may be administered in any order.

The pharmaceutical forms suitable for injectable use include sterile aqueous solutions (where water soluble) and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifingal agents, for example, parabens, chlorobutanol, phenol, sorbic acid, thimersal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, gels, pastes, viscous colloidal dispersions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound. In the case of compositions containing supplementary active ingredients, the dosages are determined by reference to the usual dose and manner of administration of the said ingredients.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; buffering agents such as sodium bicarbonate to neutralise or buffer stomach acid; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

Administration of the agent in the form of a pharmaceutical composition may be performed by any convenient means. The agent of the pharmaceutical composition is contemplated to exhibit therapeutic activity when administered in an amount which depends on the particular case. Variation depends for example, on the human or animal and the agent chosen. Dosage regimes may be adjusted to provide the optimum therapeutic response. For example, several divided doses may be administered daily, weekly, monthly or other suitable time intervals or the dose may be proportionally reduced as indicated by the exigencies of the situation. The agent may be administered in any suitable manner. Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip, patch and implant. With respect to intravenous routes, particularly suitable routes are via injection into vessels which supply the tumor or diseased organs. Peptides may also be installed into cavities for example the pleural or peritoneal cavity or injected directly into tumor tissues.

The present invention is further described with reference to the following examples which are in no way limiting on the scope of the invention.

### EXAMPLE 1

### Synthesis of Multi-Lysine Polymer 1 (MLP1)

A multi-lysine polymer (MLP1) of the formula [(NH₂-Gly)₄-Lys₂-Ser₂Lys]₅-Ala-COOH, was synthesized on an Applied Biosystems peptide synthesiser. More precisely this represents [(NH₂-Gly)₄-Lys₂-Ser₂-Lys]₄[Gly₄-Lys₂-Ser₂-Lys]Ala-COOH

More precisely the structure [(NH₂-Gly)₄-Lys₂-Ser₂-Lys]₄[Gly₄-Lys₂-Ser₂-Lys]-Ala-COOH can be represented as follows:

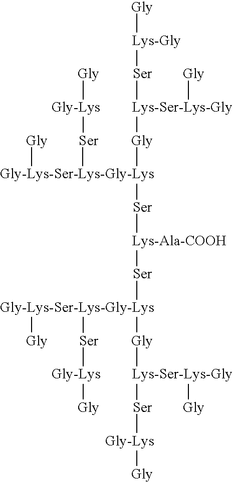

### EXAMPLE 2

### Synthesis of Multi-Lysine Polymer 2 (MLP2)

A multi-Lysine polymer (MLP2) of the general formula [(NH₂-Gly)₁₆-Lys₈-Lys₄-His₄-Glu₄-Lys₂-Lys]-Gly₅-Cys-COOH was synthesized on an Applied Biosystems peptide synthesiser. More precisely the structure can be represented as follows:

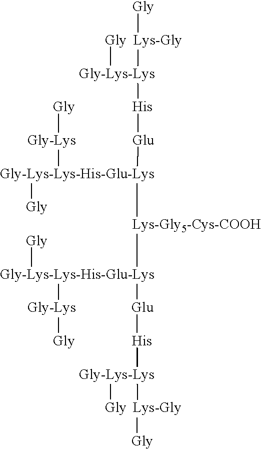

### EXAMPLE 3

### Preparation of NHS-Folate

Folic acid (5 g) was dissolved in 100 ml dry DMSO, plus 2.5 ml triethylamine. N-hydroxysuccinimide (2.6 gm) was added as a powder to the folic acid and reacted overnight with 4.7 gm dicyclohexylcarbodiimide at room temperature. The dicyclohexylurea was removed by filtration. The DMSO was concentrated under reduced pressure and heating, and NHS-folate precipitated with diethylether. The product, was washed several times with anhydrous ether, dried under vacuum and stored as a yellow powder.

### EXAMPLE 4

### Formation of MLP-Toxin Conjugates Using Biodegradable Cross-Linkers

There are many toxins which could be used for formation of folate-MLP-toxin conjugates, including momordin, psuedomonas exotoxin A, ricin and abrin.

A general method for the formation of folate-MLP-toxin conjugates is described below. Conjugates are prepared in which the covalent linker contains a biodegradable disulfide bond, which would be reduced in vivo, presumably by intracellular glutathione in the tumor cell, thereby releasing the active substance after transport from the serum into the tumor cell. Briefly, MLP1 or MLP2 was reacted with N-succinimidyl 3-(2-pyridyldithio)propionate (SPDP). The dithiopyridyl-MLP (DTP-MLP) product was purified by RP-HPLC. A free thiol was introduced onto the toxin by a two step procedure in which the toxin was firstly reacted with SPDP, after which the thiopyridyl group was with mercapto-ethanol. The product was purified by RP-HPLC. Alternatively free thiol was introduced into the toxin directly by reaction with iminothiolane. The thiolated product (SH-HN⁺toxin) was purified by RP-HPLC. Formation of the disulfide linked MLP-toxin conjugates was achieved by reaction of the thiolated toxin derivative with DTP-MLP in 2.5% acetic acid for 24 hours. The conjugated material was purified by Sephadex G-25 chromatography, followed by RP-HPLC.

Folate was linked to the polymer-toxin complexes by reacting adipyl-hydraxidylfolate derivative with the complex using EDAC. The reacted product was purified by RP-HPLC.

### EXAMPLE 5

### Synthesis of Chlorambucil-Tetra-Peptide Prodrugs

A chlorambucil-tetra peptide prodrug was synthesized that contained a sequence cleavable by intracellular lysosomal enzymes. The tetra peptide, Gly-Phe-Leu-Gly was synthesized by standard Merrifield synthesis. Chlorambucil (61 mg) and carbonyl diimidazole (32 mg) were dissolved in dry DMF (300 μl) and the solution stirred at room temperature for 1 hour. A solution of Gly-Phe-Leu-Gly (75 mg, in 300 μl DMF) was added dropwise to the active ester, followed by diisopropylethylamide (DIEA) (150 μl), and the solution was allowed to react overnight. The unreacted chlorambucil was extracted in the DCM phase of a water/DCM wash, and the Chlorambucil-Gly-Phe-Leu-Gly product was isolated from the aqueous phase by chromatography on RP-HPLC using a linear gradient of 5-100% acetonitrile.

### EXAMPLE 6

### Synthesis of Chlorambucil-Gly-Gly Prodrugs

A chlorambucil-tetra peptide prodrug was synthesized that contained a sequence that is not cleavable by intracellular lysosomal enzymes. The dipeptide, Gly-Gly was purchased from Sigma. Chlorambucil (61 mg) and carbonyl diimidazole (32 mg) were dissolved in dry DMF (300 μl) and the solution stirred at room temperature for 1 hour. A solution of Gly-Gly (75 mg, in 300 μl DMF) was added dropwise to the active ester, followed by diisopropylethylamide (DIEA) (150 μl), and the solution was allowed to react overnight. The unreacted chlorambucil was extracted in the DCM phase of a water/DCM wash, and the Chlorambucil-Gly-Gly product was isolated from the aqueous phase by chromatography on RP-HPLC using a linear gradient of 5-100% acetonitrile.

### EXAMPLE 7

### Synthesis of Cobalamin-Carboxypeptidase Conjugate

Carboxypeptidase A (Boeringher) (10 mg) was precipitated from solution by centrifugation. The resultant precipitate was resuspended in distilled water and dialysed extensively overnight against DW. The “e”monocarboxylic acid isomer of vitamin B₁₂ was dissolved at 100 mg/ml in DMF (5 mg in 50 μl). DIEA (1.5 μl) and TSTU (O-(N-succinimidyl)-N,N,N′,N′-tetramethyluronium tetrafluoroborate) (1.8 mg in 10 μl DMF) was added and allowed to stir for 10 minutes. The activated ester was then added to the carboxypeptidase A in 2% NaHCO₃ and allowed to react overnight. Unreacted vitamin B₁₂ was removed by extensive dialysis against distilled water.

1. A complex having the general formula
(TA-Q)ₙ-P-(Q′-A)ₘ
wherein, TA is selected from the group of biotin, a biotin derivative, and a biotin analog;
n is the molar substitution ratio of TA in the complex, and is a number from 1.0 to 50;
P is a pharmaceutically acceptable polymer;
A is a pharmaceutically active substance;
m is a molar substitution ratio of A in the complex, and is a number in the range from 1.0 to about 1000; and
Q and Q′ are independently a covalent bond, or a spacer compound linking TA, P and A by covalent bonds through a linkage, wherein the linkage is not a thiol-cleavable linkage.

2. The complex of claim 1, wherein the linkage is selected from the group consisting of ester, γ-glutamyl-ε-lysine, diazo, and peptide.

3. The complex of claim 1, wherein the spacer compound is a containing from two to five amino acids.

4. The complex of claim 3, wherein the peptide is selected from the group consisting of Gly-Gly, Gly-Phe-Ala, Gly-Phe-Ala-Gly, Gly-Phe-Tyr-Ala, Gly-Phe-Leu-Gly, and Gly-Phe-Tyr-Ala-Ala.

5. The complex of claim 1, wherein A is selected from the group consisting of a hormone, drug, prodrug, enzyme, protein, peptide, toxin, immunogen, DNA, DNA analog, RNA, and RNA analog.

6. The complex of claim 5, wherein the toxin is selected from the group consisting of ricin, abrin, diphtheria toxin, modecin, tetanus toxin, mycotoxins, mellitin, α-amanitin, pokeweed antiviral protein, ribosome inhibiting proteins, especially those of wheat, barley, corn, rye, gelonin and maytansinoid.

7. The complex of claim 5, wherein the toxin is a cytotoxin selected from the group consisting of an alkylating agent, an anthracycline antitumor antibiotic, an antimetabolites such as methotrexate, DON, thymidine, pentamethylmelamin, dianhydrogalactitol, 5-Methyl-THF, anguidine, maytansine, neocarzinostatin, chlorozotocin, AZQ, 2′deoxycoformycin, PALA, AD-32, m-AMSA, misonidazole, and metallopeptides comprising a metal selected from the group consisting of platinum, copper, vanadium, iron, cobalt, gold, cadmium, zinc and nickel.

8. The complex of claim 7, wherein the alkylating agent is selected from the group consisting of chlorambucil, cyclophosphamide, melphalan, cyclopropane.

9. The complex of claim 7, wherein the anthracycline antitumor antibiotic is selected from the group consisting of doxorubicin, daunomycin, adriamycin, mitomycin C, and 2-(hydroxymethyl)anthraquinone

10. The complex of claim 7, wherein the antimetabolites is selected from the group consisting of methotrexate, dichloromethatrexate: cisplatin, and carboplatin.

11. The complex, wherein A is encapsulated in a nanoparticle or microsphere vitamin complex or linked to a vitamin-polymer complex.

12. The complex of claim 1, wherein Q and Q′ are each spacer compounds independently selected from the group consisting of disuccinimidyl suberate (DSS), bis(sulfosuccinimidyl) suberate (BSS), ethylene glycolbis(succinimidylsuccinate) (EGS), ethylene glycolbis(sulfosuccinimidylsuccinate) (Sulfo-EGS), p-amino-phenylacetic acid, dithiobis(succinimidylpropionate) (DSP), 3,3′-dithiobis(sulfosuccinimidylpropionate) (DTSSP), disuccinimidyl tartarate (DST), disulfosuccinimidyl tartarate (Sulfo-DST), bis[2-(succinimidyloxycarbonyloxy)-ethylene]sulfone (BSOCOES), bis[2-(sulfosuccinimidooxycarbonyloxy)-ethylene]sulfone (Sulfo-BSOCOES), dimethyl adipimidate.2 HCl (DMA), dimethyl pimelimidate.2 HCl (DMP), dimethyl suberimidate.2 HCl (DMS), N-succinimidyl(4-iodoacetyl)aminobenzoate (SIAB), and succinimidyl 4-(p-maleimidophyl)butyrate (SMPB).

13. The complex of claim 1, wherein P is selected from a biodegradeable polymer, a non-biodegradeable polymer, and a polymer cleaved within a lysosomal vacuole.

14. The complex of claim 13, wherein the biodegradeable polymer is selected from the group consisting of dextran, a dextran derivative, and an amino acid polymer

15. The complex of claim 14, wherein the amino acid polymer is selected from the group consisting of poly-lysine and [poly-glutamic acid.

16. A method for making a complex having the general formula
(TA-Q)-P-(Q′-A)ₘ
wherein, TA is selected from the group of biotin, biotin derivative, and biotin analog;
n is the molar substitution ratio of TA in the complex, and is a number from 1.0 to 50;
P is a pharmaceutically acceptable polymer;
A is a pharmaceutically active substance;
m is a molar substitution ratio of A in the complex, and is a number in the range from 1.0 to about 1000;
Q and Q′ are independently a covalent bond, or a spacer compound linking TA, P and A by covalent bonds; and
wherein TA, P, and A are joined by a linkage, wherein the linkage is not a thiol-cleavable linkage comprising one or more of the following steps:
(a) reacting A with P to form an intermediate A-P complex;
(b) chemically modifying the A to provide at least one functional group capable of forming a chemical linkage, and reacting A and P to form the intermediate A-P complex;
(c) chemically modifying TA to provide at least one functional group capable of forming a chemical linkage and reacting TA and P to form an intermediate TA-P complex;
(d) chemically modifying A and P to provide functional groups capable of forming a chemical linkage, and reacting A and P to form an intermediate A-P complex;
(e) reacting A with at least one cross-linking agent and reacting A with P to form a intermediate A-P complex;
(f) reacting TA with at least one cross-linking agent and reacting P and a carrier to form an intermediate TA-P complex;
(g) reacting A and P with at least one cross-linking agent and reacting the A and P to form an intermediate A-P complex;
(h) reacting the A directly with a polymeric support to form an intermediate containing one or more molecules of the A linked to P to form a support-bound A-P intermediate complex, and subsequently coupling the support-bound A-P intermediate complex to one or more TA; and
(i) coupling one or more TA to a polymeric support and subsequently reacting support-bound TA intermediate with one or more molecules of the A to yield a complex containing one or more molecules of A.

17. A pharmaceutical composition comprising
(i) the complex of claim 1, and
(ii) a pharmaceutically acceptable carrier, excipient, diluent or adjuvant or a combination thereof.

18. A method of making the pharmaceutical composition of claim 16, comprising mixing the complex of claim 1 together with a pharmaceutically acceptable carrier, excipient, diluent or adjuvant or a combination thereof.

19. A method for the treatment, prophylaxis or amelioration of a disease, comprising administering to a subject in need thereof a therapeutically effective amount of a complex having the general formula
(TA-Q)ₙ-P-(Q′-A)ₘ
wherein, TA is selected from the group of biotin, biotin derivative, and biotin analog;
n is the molar substitution ratio of TA in the complex, and is a number from 1.0 to 50;
P is a pharmaceutically acceptable polymer;
A is a pharmaceutically active substance;
m is a molar substitution ratio of A in the complex, and is a number in the range from 1.0 to about 1000;
Q an d Q′ are independently a covalent bond, or a spacer compound linking TA, P and A by covalent bonds; and
wherein TA, P, and A are joined by a linkage, wherein the linkage is not a thiol-cleavable linkage.

20. The method of claim 19, wherein the disease is cancer.

21. The method of claim 19, wherein the subject is a vertebrate host selected from the group consisting of a veterinary animal, a domestic animal, an agricultural animal, and a human.

22. The method of claim 19, wherein the administration is parenteral administration.

23. A kit for the delivery of one or more agents to a subject comprising the complex of claim 1.
